(19) United States
(12) Patent Application Publication
(10) Pub. No.: US 2010/0233162 A1
(43) Pub. Date: Sep. 16, 2010

(54) LOCAL ADMINISTRATION OF CHICKEN YOLK IMMUNE GLOBULINS (IgY) TO TREAT AND PREVENT FUNGAL INFECTIONS

(75) Inventors: Anders Larsson, Uppsala (SE); Hans Kollberg, Uppsala (SE)

Correspondence Address: SCHWEGMAN, LUNDBERG & WOESSNER, P.A., P.O. BOX 2938, MINNEAPOLIS, MN, 55402, US

(73) Assignee: Bengt AGERUP, Uppsala (SE)

(21) Appl. No.: 12/673,379

(22) PCT Filed: Aug. 28, 2008
(86) PCT No.: PCT/EP2008/061309

§ 371 Date: Mar. 22, 2010

Related U.S. Application Data
(60) Provisional application No. 60/968,893, filed on Aug. 30, 2007.

(30) Foreign Application Priority Data
Aug. 30, 2007 (SE) 0701952-4

(51) Int. Cl. A61K 39/395; A61K 39/00; G01N 33/53; A61P 3/10; A61P 1/00; A61P 11/00; A61P 15/00

(52) U.S. Cl. 424/133.1; 424/172.1; 424/152.1; 424/274.1; 435/7.1

(57) ABSTRACT

The present invention relates to a composition comprising IgY antibodies against at least two different fungi, the use of the composition for the preparation of a pharmaceutical especially for prophylaxis and/or therapy of all kinds of fungal infections, such as conditions caused by organisms belonging to the Candida genus or Aspergillus genus, especially of fungi that are less sensitive to antimycotica and a diagnotstic method using the IgY antibodies. Further it relates to immunisation of birds with a combination of at least two different species of fungi to the Candida genus or Aspergillus genus and a diagnostic method for fungi. It also relates to relates to a novel method for treatment and/or prophylaxis of fungal infections, especially infections by fungi which have lowered sensitivity or resistance to antimycotics. The method is both safe and effective. This is achieved by using IgY directed against fungi, in particular against Candida species, which have a lowered sensitivity or resistance against antimycotics. The specific IgY has been obtained by hyper immunising birds with one or several fungi as antigen in order to stimulate the production of immune globulins (IgY) against such fungi. The present invention also relates to a pharmaceutical product from eggs of birds containing immune globulin or a fragment thereof, which can be combined with other preparations, nutritients or pharmaceuticals for simultaneous, separate or sequential use in the prophylaxis or therapy of gastrointestinal infections in newborn infants.

### FIELD OF INVENTION

The present invention relates to a composition comprising IgY antibodies against at least two different fungi, the use of the composition for the preparation of a pharmaceutical especially for prophylaxis and/or therapy of all kinds of fungal infections, such as conditions caused by organisms belonging to the Candida genus or Aspergillus genus, especially of fungi that are less sensitive to antimycotica. Immunisation of birds with a combination of at least two different species of fungi is also envisaged.

It also relates to a method for prophylaxis or therapy of all kinds of fungal infections—e.g. candida species, aspergillus species, etc.—in any human, who has problems with these kinds of diseases and a diagnostic method using the IgY antibodies. Further it relates to a diagnostic method for fungi.

### BACKGROUND OF THE INVENTION

Fungal infections might be recurrent, severe and for some patients life threatening.—e.g. patients with APS1, patients treated with corticosteroids or immune suppressants, newborn and/or prematurely born infants with an immature immune system, patients suffering from temporary immune deficiency and immune deficiency diseases such as congenital immune deficiencies and AIDS, elderly patients with decline of their immune system, patients treated with antibiotics, and others. An effective immune system is of outmost importance to fight infections. The production of immune globulin is very immature in newborn infants. They have to rely solely on immune globulins transported to them from their mother via placenta and there is no immune globulin transported before the 32^{nd} week of pregnancy. The immune system is seriously damaged in patients with cancer and AIDS and in patients treated with immune suppressants. Fungal infections are common in patients treated with antibiotics, since their normal flora—in the gastro-intestinal tract, in the respiratory tract and in the vagina—is disturbed and thereby their natural barrier against bacteria and fungi is damaged. Use of antibiotics changes this natural barrier, which means that fungi have free access to the epithelial membranes. Fungal infections in these already damaged epithelia are very difficult to treat with antimycotics.

The only treatment of fungal infections that exists today is to use antimycotics. Treatment with antimycotics has many drawbacks. The most alarming is that several fungi very rapidly become resistant against these drugs. Repetitive use of antimycotics is therefore undesirable. Antimycotics have very often to be combined with immune suppressive drugs, and are then often very toxic especially for the kidneys and for the bone marrow. Newborn infant is more susceptible to these toxic effects than older children and adults.

The natural response by humans to any infection is the production of antibodies of the IgG-class—directed to systemic infections—and secretory IgA—directed to infections of the mucus membranes, including those in the oral cavity, the digestive tract, the respiratory tree and genitourinary tract. When the immune system is not capable to produce sufficient amount or specific types of antibodies, the ability to use an extraneous source of immune globulins in the treatment and prophylaxis for these patients is an attractive alternative.

Conventional extraneous sources of bulk polyclonal antibodies have mainly been derived from serum of mammals after having immunised the mammal with a specific microbe. The production of IgG antibodies requires a large number of animals, which have to be immunised and bled repeatedly. The polyclonal antibodies are antigenic in them selves. Due to this they will raise anti-IgG antibodies against them selves, which will cause loss of their activity. They are therefore unsuitable for long-term application in humans.

The new idea of this patent is to derive the polyclonal antibodies from immunised hens. Immune globulin from hens (IgY) has several advantages over mammalian IgG. Thus there are also ethical and animal protection objections to the use of IgG. Orally administered IgY does not raise any anti-IgY antibodies. Thus IgY will not loose activity even after having been used for a long time.

In view of the drawbacks associated with prior art methods for treatment and prophylaxis of fungal infections in patients, who are prone to get recurrent, severe and even life threatening such infections, there is a need for a new and improved method for treatment and prophylaxis of such infections.

### SUMMARY OF THE INVENTION

The present invention relates to a composition comprising IgY antibodies against at least two different fungi, the use of the composition for the preparation of a pharmaceutical especially for prophylaxis and/or therapy of all kinds of fungal infections, such as conditions caused by organisms belonging to the Candida genus or Aspergillus genus, especially of fungi that are less sensitive to antimycotica and a diagnostic method using the IgY antibodies. Immunisation of birds with a combination of at least two different species of fungi is also envisaged. Further it relates to a diagnostic method for fungi.

It especially relates to a novel method for treatment and prophylaxis of such fungal infections, which is both safe and effective. This is achieved by using IgY directed against two or more fungi—in particular against Candida albicans—, which has been obtained from the egg yolk of birds, that have been hyper immunised with these fungi in order to stimulate the production of immune globulins (IgY) against them.

The conventional way of treating said patients has been by using antimycotics. This way has several disadvantages. The applicants' intensive studies of IgY have led them to the surprising discovery that a composition comprising IgY from hens raised against two or more fungi gives a very efficient avian IgY for treatment of fungal infections in patients with a high risk of such infections without any risk of developing resistant fungi. In vitro tests have shown, that the IgY against fungi also protects against fungi that have a lowered sensitivity to and even are resistant to antimycotics. No one has previously considered this possibility.

The present invention also relates to a pharmaceutical product from eggs of birds containing immune globulin or a fraction thereof, which can be combined with other preparations or pharmaceuticals for simultaneous, separate or sequential use in the prophylaxis or therapy of fungal infections.

The present invention further relates to a pharmaceutical IgY product including any other nutritional agent, including human breast milk or a substitute therefore. The present invention also pertains to a prophylaxis or therapy method including administration of IgY together with any nutritional agent.

The present invention further relates to a pharmaceutical IgY product including a buffering agent. The present invention also pertains to a prophylaxis or therapy method including administration of IgY together with such a buffering agent.

The present invention relates to prophylaxis or therapy of all kind of fungal infections in patients, who are prone to get recurrent, severe and even life threatening such infections. The infection can be any infection caused by a fungus and most innovative by fungi that have a lowered sensitivity to or resistance to antimycotics—preferably infections caused by Candida species.

The invention is described by the following figures.

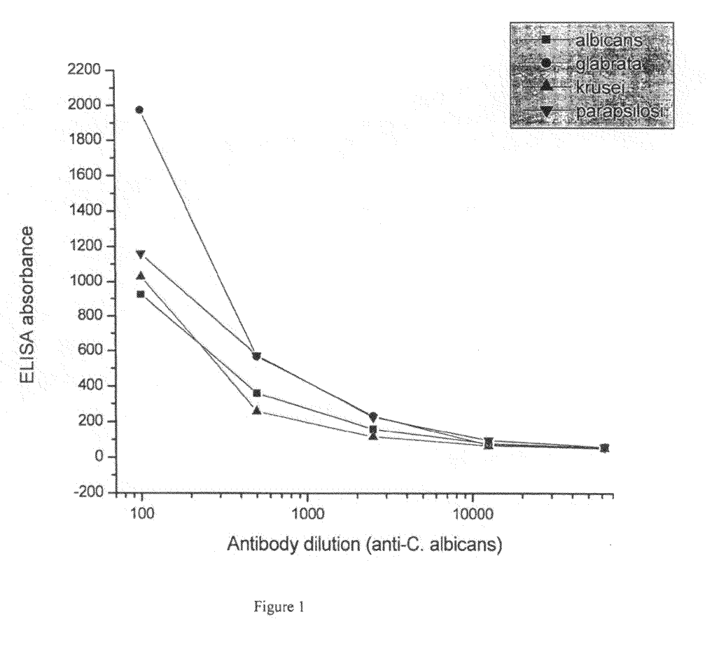

FIG. 1 is a graph of absorbance against dilution of antibodies against Candica albicans obtained in an ELISA in vitro test for activity against Candida albicans, Candida glabrata, Candida krusei and Candida parapsilosi.

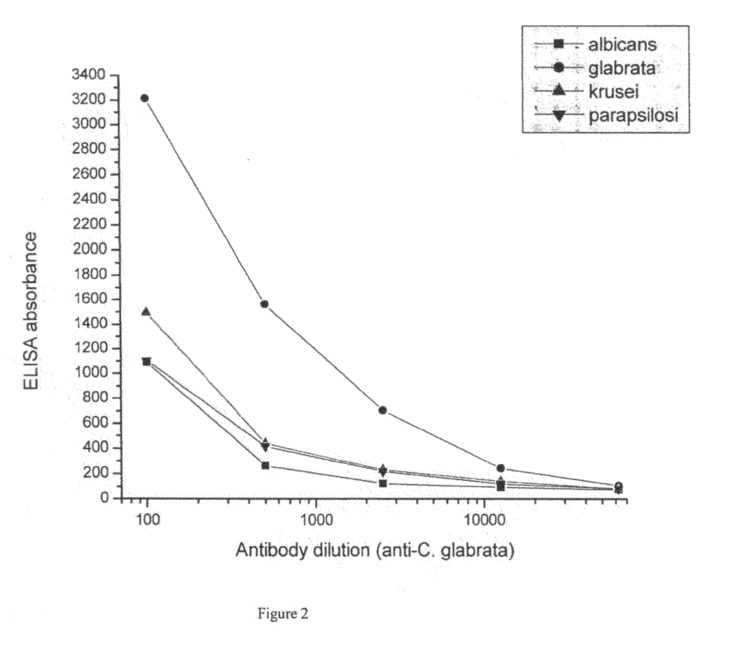

FIG. 2 is a graph of absorbance against dilution of antibodies against Candida glabrata obtained in an ELISA in vitro test for activity against Candida albicans, Candida glabrata, Candida krusei and Candida parapsilosi.

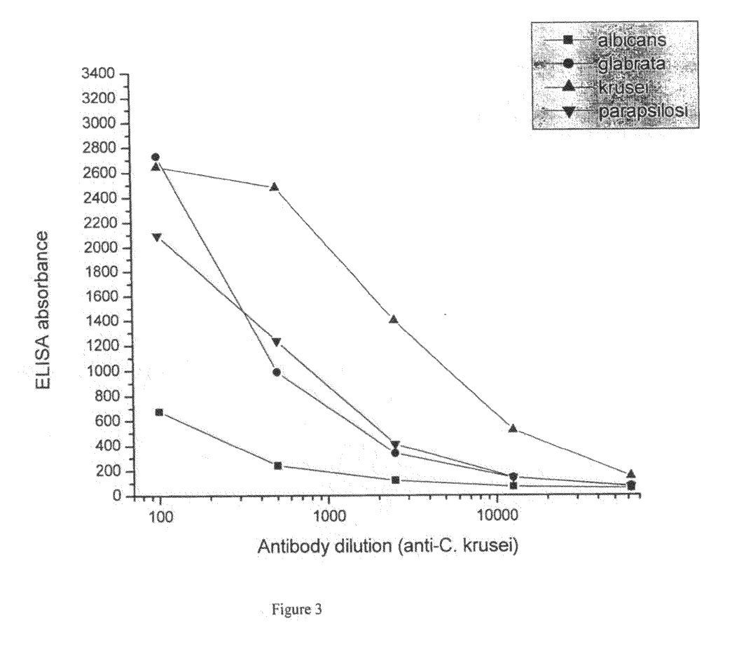

FIG. 3 is a graph of absorbance against dilution of antibodies against Candida krusei obtained in an ELISA in vitro test for activity against Candida albicans, Candida glabrata, Candida krusei and Candida parapsilosi.

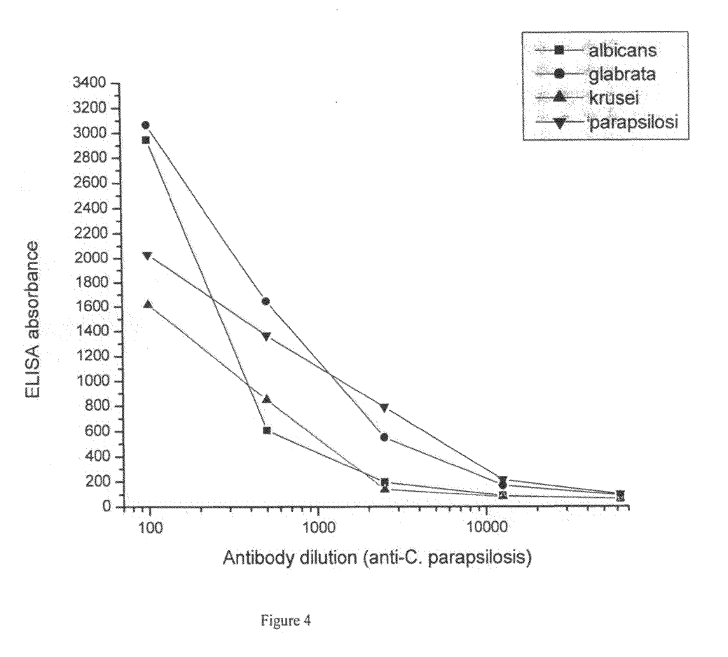

FIG. 4 is a graph of absorbance against dilution of antibodies against Candida parapsilosi obtained in an ELISA in vitro test for activity against Candida albicans, Candida glabrata, Candida krusei and Candida parapsilosi.

### DETAILED DESCRIPTION IF THE INVENTION

The present invention relates to a composition comprising IgY antibodies against at least two different fungi chosen from Candida albicans, Candida dubliniensis, Candida glabrata, Candida guilliermondii, Candida kefyr, Candida krusei, Candida lusitaniae, Candida milleri, Candida oleophila, Candida parapsilosis, Candida tropicalis and Candida utilis Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor.

The present invention is based on the discovery that anti-candida IgY, derived from birds, such as hens vaccinated against two or more species of candida or other fungi, has an unexpectedly good capacity to prevent and/or cure infections from such fungi even if these fungi have a lowered sensitivity or even resistance to antimycotics.

IgY is the bird antibody such as the chicken antibody that corresponds to mammalian IgG. IgY consists of two light chains and two heavy chains and has a molecular weight of approximately 180 000 Da. IgY is actively transported from the hen to the egg and the egg yolk, which thus contains high concentrations of IgY.

The IgY may derive from any bird such as galliform and non-galliform birds. Examples of galliformes are turkeys, grouse, chickens, quails, and pheasants. Non-galliforms may be ducks.

One egg yolk contains around 100-200 mg of IgY antibodies. Most humans regularly consume ½-1½ egg per day and have achieved tolerance against proteins from eggs (including the immune globulin from eggs (IgY)). These patients will not get any allergic reaction when treated orally with IgY. Thus, there is no risk for an allergic response when treating these patients orally with IgY. However, patients with known egg allergy should not be treated with IgY. A dose in the order of 2 mg IgY would probably suffice to achieve the desired prophylactic or curing effects.

Hens, which have been immunised with microbes, respond by producing specific, polyclonal antibodies against the microbe. The antibodies can be purified from the egg yolk. Several in vitro studies show that bacterial, viral and fungal infections can be prevented with IgY. Many studies have also shown that per oral administration of specific IgY is used successfully to treat bacterial, viral and fungal infections in animals. However, no one has shown that specific IgY can also be active against fungi resistant to antimycotics.

As IgY is a normal dietary component there is no risk of toxic reactions in the patient, except for those who have a known allergy to eggs.

Compared to mammalian polyclonal antibodies IgY reacts with different epitopes on the antigen than the mammalian antibodies do. This gives access to a different antibody repertoire than the mammalian antibody. The mode of action of the specific antibody is related to the number of organisms present at a given moment. It will be appreciated that there is a direct molecular correlation between antibody entities attaching to each microbe and the numbers of microbes present. The dose level will also be related to the total surface area of affected tissue and biological parameters, which affect “wash out” ratios.

Egg immune globulin is classified as avian IgY, which is similar to mammalian secretory IgA, and therefore a natural part of the mucus epithelial environment.

One of the objects according to the invention is to provide a pharmaceutical composition from eggs of birds comprising immune globulin or a fraction thereof for use in the prophylaxis or therapy of fungal infections.

As indicated above, fungal infections can be attributed to a multiplicity of factors, including long-term exposure to antibiotics, which disrupt the normal balance of the intestinal micro-flora. The preparation will be designed to reinstitute the normal balance of the micro flora. The formulations according to the present invention can be used as an alternative or supplement to antimycotic treatments.

According to one embodiment the invention comprises a composition comprising immuneglobuline Y (IgY) antibodies against at least two different fungi. The fungi may be chosen from Candica genus and the Aspergillus genus.

According one embodiment the composition may comprise IgY antibodies against at least two, at least three, at least four different fungi.

Suitable species from the Candida genus are Candida albicans, Candida dubliniensis, Candida glabrata, Candida guilliermondii, Candida kefyr, Candida krusei, Candida lusitaniae, Candida milleri, Candida oleophila, Candida parapsilosis, Candida tropicalis and Candida utilis. According one embodiment the composition may comprise IgY antibodies against at least two, at least three, at least four of the above mentioned Candida species. The composition may comprise 2-12, 2-11, 2-10, 2-9, 2-8, 2-7, 2-6, 2-5, 2-4 and 2-3 species of the Candida genus such as of the above mentioned Candida species. Thus, the composition may comprise IgY antibodies against 2, 3, 4, 5, 6, 7, 8, 9, 10, 11 and 12 of the above mentioned Candida species.

Suitable species from the Aspergillus genus are species causing aspergillosis, such as, Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor. According one embodiment the composition may comprise IgY antibodies against at least two, at least three, at least four of the above mentioned Aspergillus species. The composition may comprise 2-10, 2-9, 2-8, 2-7, 2-6, 2-5, 2-4 and 2-3 species of the Aspergillus genus such as of the above mentioned Aspergillus species. Thus, the composition may comprise IgY antibodies against 2, 3, 4, 5, 6, 7, 8, 9 and 10 of the above mentioned Aspergillus species.

According one embodiment the composition may comprise IgY antibodies against at least two, at least three, at least four of the above mentioned Aspergillus and Candida species. The composition may comprise 2-22, 2-21, 2-20, 2-19, 2-18, 2-17, 2-16, 2-15, 2-14, 2-13, 2-12, 2-11, 2-10, 2-9, 2-8, 2-7, 2-6, 2-5, 2-4 and 2-3 species of the Candida and Aspergillus genus such as of the above mentioned Candida and Aspergillus species. Thus, the composition may comprise IgY antibodies against 1, 2, 3, 4, 5, 6, 7, 8, 9, 10, 11 and 12 of the above mentioned Candida species together with 1, 2, 3, 4, 5, 6, 7, 8, 9 and 10 of the above mentioned Aspergillus species.

According to one aspect of the invention one of the species is Candida albicans.

According to another aspect of the invention the at least two species are chosen from Candida albicans, Candida glabrata, Candida krusei and Candida parapsilosis. Thus, the composition may comprise 2, 3 and all of these Candida species.

According to a further aspect of the invention the composition comprises at least two such as 2-4 of Candida albicans, Candida glabrata, Candida krusei and Candida parapsilosis together with 1, 2, 3, 4, 5, 6, 7, 8, 9, 10, 11, 12, 13, 14, 15, 16, 17 and 18 of the other of the above mentioned Candida and Aspergillus species mentioned above, such as 1, 2, 3, 4, 5, 6, 7, 8 of the above mentioned Candida species and/or 1, 2, 3, 4, 5, 6, 7, 8, 9, 10 of the above mentioned Aspergillus species mentioned above.

The antibodies according to the invention may be polyclonal antibodies, monoclonal antibodies (mAbs), chimeric antibodies, anti-idiotypic (anti-Id), humanised abtibodies and antibodies to antibodies that can be labelled in soluble or bound form, as well as fragments thereof, such as, for example, Fab and F(ab′)2—fragments lacking the Fc fragment of intact antibody, which are capable of binding antigen.

By the terms “immune globulin” or “fragment of an immune globulin” is meant an antibody, or antibody fragment, or antibody precursor capable of binding to a specific microbe or fragment thereof so as to render it non-pathogenic.

Polyclonal IgY antibodies may be produced by immunising hens with a fungi antigen of interest as described e.g. in U.S. Pat. No. 5,367,054 and in example 1 below.

The expression “monoclonal antibody” is art-recognized terminology. The IgY monoclonal antibodies of the present invention can be prepared using classical cloning and cell fusion techniques. The immunogen (antigen) of interest, e.g., a suspension of a Candida species fungi of interest, is typically administered (e.g., intraperitoneal injection) to hens to induce an immune response. The hen may be boosted, for example, three or four times, lymphoid cells obtained from lymphoid organs such as thymus, bursa fabricius, lymphoid nodules, bone marrow or spleen, e.g. splenocytes and are extracted and fused with myeloma cells using the well-known processes of Kohler and Milstein (Nature 256: 495-497 (1975)) and Harlow and Lane (Antibodies: A Laboratory Manual, Cold Spring Harbor Laboratory, New York, 1988). The resulting hybrid cells are then cloned in the conventional manner, e.g., using limiting dilution, and the resulting clones, which produce the desired IgY monoclonal antibodies, cultured.

A humanized antibody is and antibody obtained by modifying an antibody heterogeneous to a human being such as a mouse antibody to replace its primary structure other than CDR (complementary-determining region) of H chain and L chain with the corresponding primary structure of a human antibody. Methods for producing them and references are e.g. described in U.S. Pat. No. 6,645,734.

The composition according the invention may further comprise pharmaceutically acceptable carriers, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispensing agents, nutritional agents, e. g. human breast milk or a substitution thereof, functional foods, probiotica and bacteria that are not harmful and against which the IgY antibodies are not directed.

When the immune globulin is to be administered by the oral route, it will preferably contain a buffering agent to prevent deactivation at low pH-values, which can optionally be administered in the form of a nutritional complement.

IgY antibodies also have biochemical properties that make them advantageous over IgG for per oral immunotherapy: They neither activate the human complement system nor react with rheumatoid factors, human anti-mouse IgG antibodies (HAMA) or human Fc-receptors. Those are all well-known cell activators and mediators of inflammation.

According to a basic embodiment of the present invention, by mixing the IgY according to the present invention with any pharmaceutically acceptable carrier or diluents, a pharmaceutical composition or medicament is obtained. The medicament containing IgY can be formulated as a freeze-dried or lyophilised powder, a solution, a lozenge, a tablet, a capsule, an ointment, a crème, a vagitorium or a suppositorium.

Probiotics are defined as live micro-organisms, including Lactobacillus species, Bifidobacterium species and yeasts that may beneficially affect the host upon ingestion by improving the balance of the intestinal micro flora.

Bifidobacterium are also classified as lactic acid bacteria (LAB). Bifidobacteria used as probiotics include Bifidobacterium adolescentis, Bifidobacterium bifidum, Bifidobacterium animalis, Bifidobacterium thermophilum, Bifidobacterium breve, Bifidobacterium longum, Bifidobacterium infantis and Bifidobacterium lactis. Specific strains of bifidobacteria used as probiotics include Bifidobacterium breve strain Yakult, Bifidobacterium breve RO7O, Bifidobacterium lactis Bb12, Bifidobacterium longum RO23, Bifidobacterium bifidum RO71, Bifidobacterium infantis RO33, Bifidobacterium longum BB536 and Bifidobacterium longum SBT-2928.

Lactobacilli are also classified as lactic acid bacteria (LAB). Lactobacilli used as probiotics include Lactobacillus acidophilus, Lactobacillus brevis, Lactobacillus bulgaricus, Lactobacillus casei, Lactobacillus cellobiosus, Lactobacillus crispatus, Lactobacillus curvatus, Lactobacillus fermentum, Lactobacillus GG (Lactobacillus rhamnosus or Lactobacillus casei subspecies rhamnosus), Lactobacillus gasseri, Lactobacillus johnsonii, Lactobacillus plantarum and Lactobacillus salivarus. Lactobacillus plantarum 299v strain originates from sour dough. Lactobacillus plantarum itself is of human origin. Other probiotic strains of Lactobacillus are Lactobacillus acidophilus BG2FO4, Lactobacillus acidophilus INT-9, Lactobacillus plantarum ST31, Lactobacillus reuteri, Lactobacillus johnsonii LA1, Lactobacillus acidophilus NCFB 1748, Lactobacillus casei Shirota, Lactobacillus acidophilus NCFM, Lactobacillus acidophilus DDS-1, Lactobacillus delbrueckii subspecies delbrueckii, Lactobacillus delbrueckii subspecies bulgaricus type 2038, Lactobacillus acidophilus SBT-2062, Lactobacillus brevis, Lactobacillus salivarius UCC 118 and Lactobacillus paracasei subsp paracasei F19.

Lactococci are gram-positive facultative anaerobes. They are also classified as lactic acid bacteria (LAB). Lactococcus lactis (formerly known as Streptococcus lactis) is found in dairy products and is commonly responsible for the souring of milk. Lactococci that are used or are being developed as probiotics include Lactococcus lactis, Lactococcus lactis subspecies cremoris (Streptococcus cremoris), Lactococcus lactis subspecies lactis NCDO 712, Lactococcus lactis subspecies lactis NIAI 527, Lactococcus lactis subspecies lactis NIAI 1061, Lactococcus lactis subspecies lactis biovar diacetylactis NIAI 8 W and Lactococcus lactis subspecies lactis biovar diacetylactis ATCC 13675.

Saccharomyces belongs to the yeast family. The principal probiotic yeast is Saccharomyces boulardii. Saccharomyces boulardii is also known as Saccharomyces cerevisiae Hansen CBS 5296 and S. boulardii. S. boulardii is normally a nonpathogenic yeast. S. boulardii has been used to treat diarrhea associated with antibiotic use.

Lactococci are gram-positive facultative anaerobes. They are also classified as lactic acid bacteria (LAB). Lactococcus lactis (formerly known as Streptococcus lactis) is found in dairy products and is commonly responsible for the souring of milk. Lactococci that are used or are being developed as probiotics include Lactococcus lactis, Lactococcus lactis subspecies cremoris (Streptococcus cremoris), Lactococcus lactis subspecies lactis NCDO 712, Lactococcus lactis subspecies lactis NIAI 527, Lactococcus lactis subspecies lactis NIAI 1061, Lactococcus lactis subspecies lactis biovar diacetylactis NIAI 8 W and Lactococcus lactis subspecies lactis biovar diacetylactis ATCC 13675.

Saccharomyces belongs to the yeast family. The principal probiotic yeast is Saccharomyces boulardii. Saccharomyces boulardii is also known as Saccharomyces cerevisiae Hansen CBS 5296 and S. boulardii. S. boulardii is normally a nonpathogenic yeast. S. boulardii has been used to treat diarrhea associated with antibiotic use.

Streptococcus thermophilus is a gram-positive facultative anaerobe. It is a cytochrome-, oxidase- and catalase-negative organism that is no motile, non-spore forming and homofermentative. Streptococcus thermophilus is an alpha-hemolytic species of the viridans group. It is also classified as a lactic acid bacteria (LAB). Steptococcus thermophilus is found in milk and milk products. It is a probiotic and used in the production of yogurt. Streptococcus salivarus subspecies thermophilus type 1131 is another probiotic strain.

The above probiotic bacteria are useful according to the invention. Lactobacilli, such as Lactobacillus reuteri are used according to one embodiment.

The invention also regards a composition and the use of a composition comprising IgY antibodies against at least two species from fungi chosen from Candida albicans, Candida dubliniensis, Candida glabrata, Candida guilliermondii, Candida kefyr, Candida krusei, Candida lusitaniae, Candida milleri, Candida oleophila, Candida parapsilosis, Candida tropicalis and Candida utilis Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor for the preparation of a pharmaceutical.

Further, the invention relates to a composition and the use of a composition comprising IgY antibodies against at least two different fungi chosen from Candida albicans, Candida dubliniensis, Candida glabrata, Candida guilliermondii, Candida kefyr, Candida krusei, Candida lusitaniae, Candida milleri, Candida oleophila, Candida parapsilosis, Candida tropicalis and Candida utilis Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor for the preparation of a pharmaceutical for profylax and/or treatment of a condition caused by fungi, such as organisms belonging to the Candida genus or Aspergillus genus, especially of fungi that are less sensitive (i.e. resistant) to antimycotica.

Moreover, the invention relates to a method for treatment of a condition caused by an organism belonging to the Candida genus and/or the Aspergillus genus wherein a composition comprising IgY antibodies against at least two fungi is administrated to an individual in need thereof. The individual may be a mammal and especially a human being.

The invention also relates to a method for immunisation of birds with a combination of at least two different species of fungi chosen from the above mentioned Candida and Aspergillus species. All specifications of types of birds, fungi and intervals of figures and enumeration of figures apply for all different aspects of the invention. Thus, for example the above stated types of fungi and intervals of figures and enumeration of figures apply also for the immunisation of birds of the different types mentioned above.

The condition caused by Candida may conditions caused by Candida overgrowth chosen from dysfunction in glands and organs, such as adrenal and thyroid gland malfunction, cold hands or feet, diabetes mellitus, hypoglycemia, hypothyroidism, impotence, low body temperature; gastrointestinal dysfunction such as bad breath (halitosis), bloating, coating on tongue (oral thrush), constipation, diarrhea, dry mouth, gas, heartburn, indigestion, irritable bowel syndrome, obesity or/and excessive weight loss; psychological and Allergic dysfunctions such as acne, Blurred vision, bronchitis (recurrent), burning or tingling, chemical sensitivity, chest pain, coughing, earaches, hayfever, headaches, hives, muscle aches, pain, weakness and tension, nasal congestion, head tension, numbness, painful, swollen, stiff joints, shortness of breath, sinusitis, sore throats; emotional and mental dysfunctions chosen from ADD, ADHD, anxiety, depression, disorientation, drowsiness, fatigue, feelings of unreality, frequent mood swings, hyperactivity, inability to concentrate, insomnia, irritability, low energy, mental confusion, MS, nervousness, poor memory: skin dysfunctions such as Acne, anal itch, athlete's foot, dandruff, dermatitis, diaper rash, dry skin, eczema, excessive perspiration, facial rash, fungal infection of the nails, hives, Impetigo, jock itch, lupus, psoriasis, tingling and numbness; genitourinary dysfunction chosen from bladder infection (recurrent), burning on urination, cramps, cystis, endometriosis (irregular or painful menstruation), fluid retention (oedema), frequent urination, impotency, infertility, loss of sexual feelings, menstrual irregularities, painful intercourse, PMS, prostatitis, recurrent yeast vaginitis, vaginal burning, itching or discharge.

The condition caused by the Aspergillus genus may be aspergillosis such as allergic bronchopulmonary aspergillosis, pulmonary aspergilloma and invasive aspergillosis and colonization of bacteria from the Aspergillus genus in the respiratory tract. Examples of forms of the disease are colonization of sinuses, and lungs; toxicoses, allergic bronchopulmonary aspergillosis in sinuses and lungs; pulmonary aspergilloma, invasive aspergillosis—pulmonary aspergillosis 1;—CNS—aspergillosis, sinonasal aspergillosis, Ooteomyelitis, endophthalmitis, endocarditis, renal abscesses, cutaneous: burns, post surgical wounds, otomycosis, exogenous endophthalmitis, allergic fungal sinusitis and urinary tract fungus balls.

The pharmaceutical product according to the present invention can also be used in conjunction with, or include, an antimicrobial agent of the kind used in conventional therapy of infections.

The products and methods of IgY for prophylaxis or treatment according to the present invention have also been working on patients suffering from temporary immunodeficiency, e.g. immune suppressed patients with leukaemia have been treated successfully with anti-candida IgY. Said administration relates to oral application in order to prevent or treat oral and pharyngeal candidiasis infections caused by Candida albicans. Said type of administration for the purpose of preventing or treating oral and pharyngeal infections is disclosed in another application filed previously by the same inventors is not part of the subject matter of the present invention. However, early studies of administering anti-candida IgY to immune suppressed patients in order to remove candida from the gastrointestinal canal in order to avoid enteric infections and infections in other parts of the body, such as the vagina, seems promising. In order to treat enteric infections, the anti-candida IgY needs to be buffered or combined with a nutritional agent according to the present invention, if not administered to a newborn or a prematurely born infant. Thus, the present invention also relates to prophylaxis or treatment of patients with temporary immunodeficiency and immunodeficiency diseases such as AIDS.

This pharmaceutical medicament is preferably administered for administration orally, locally on the skin, in rectum or in vagina or by inhalation together with any other pharmaceutically acceptable carrier or diluents.

Formulations suitable for oral administration” means formulations which are in a form suitable to be administered orally to a patient. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste. This pharmaceutical medicament is administered for administration orally, locally on the skin, in rectum or in vagina or by inhalation together with any other pharmaceutically acceptable carrier or diluents.

In another embodiment the pharmaceutical medicament according to the present invention is formulated as a controlled or sustained release formulation.

According to another embodiment of the present invention, the IgY can be administered without any conventional diluents or recipient in a nutritional agent such as human breast milk or a substitute therefore.

During their experiments, the present inventors have found that the combination of human breast milk and IgY, administered as an emulsion, protects the IgY and conserves its activity. The present inventors believe that this is due to the emulsion that is formed from IgY and the milk. This can also be an effect of the buffering ability of the milk, which enhances the lifetime of the IgY.

Formulations suitable for nasal or inhalational administration means formulations which are in a form suitable to be administered nasally or by inhalation to a patient. The formulation may contain a carrier, in a powder form, having a particle size for example in the range l to 500 microns (including particle sizes in a range between 20 and 500 microns in increments of 5 microns such as 30 microns, 35 microns, etc.) Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents. Inhalational therapy is readily administered by metered dose inhalers.

Formulations suitable for rectal administrations means formulations which are in a form suitable to be administered rectally to a patient. The formulation is preferably in the form of suppositories which can be prepared by mixing the compounds useful according to this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

Formulations suitable for vaginal administration means formulations which are in a form suitable to be administered vaginally to a patient. The formulation may be presented as pessaries, tampons, creams, crels, pastes, foams or spray formulations containing in addition to the active ingredient such carriers as are known in the art to be appropriate.

“Formulations suitable for local or topical administration means formulations which are in a form suitable to be administered topically to a patient. The formulation may be presented as a topical ointment, salves, powders. sprays and inhalants, gels (water or alcohol based), creams, as is generally known in the art, or incorporated into a matrix base for application in a patch, which would allow a controlled release of compound through the transdermal barrier, When formulated in an ointment, the active ingredients may be employed with either a paraffinic or a water-miscible ointment base.

In another embodiment the pharmaceutical medicament according to the present invention is formulated as a controlled or sustained release formulation.

According to another embodiment of the present invention, the IgY can be administered without any conventional diluents or recipient in a nutritional agent such as human breast milk or a substitute therefore.

The amount of antibody or fragment thereof administered or the schedule for administration will vary among individuals based on age, size, weight, condition, the mode of administration, the diagnosis and the severity of the condition to be treated. One skilled in the art will realize that dosages are best optimized by the practicing physician and methods for determining dosage are described, for example in Remington's Pharmaceutical Science, 16.sup.th ed., 1980, Mack Publishing Co., edited by Oslo et al. Guidance in selecting appropriate doses for antibodies is found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389.

A typical dose of the antibody used alone might range from about 1 .mu.g/kg to up to 100 mg/kg of body weight or more per day, and preferably 1 .mu.g/kg to up to 1 mg/kg, depending on the factors mentioned above.

Advantageously, the pharmaceutical compositions for oral or parenteral use described above are prepared into pharmaceutical preparations with a unit dose to fit a dose of the active ingredients. Such unit dose preparations include, for example, tablets, pills, capsules, injections (ampoules) and suppositories. The amount of the antibody contained is generally about 5 to about 500 mg per dosage unit form; it is preferred that the aforesaid antibody is contained in about 5 to about 100 mg especially in the form of injection, and in about 10 to 250 mg for the other forms.

The therapeutic/preventive agent comprising the antibody of the present invention can be administered orally or parenterally to human or mammals (e.g., rats, rabbits, sheep, swine, bovine, cats, dogs, monkeys, etc.). The dose may vary depending on subject to be administered, target disease, symptoms, route for administration, etc. When used parenterally the agend may be administered in a single dose of normally about 0.01 to 20 mg/kg body weight, preferably about 0.1 to 10 mg/kg body weight and more preferably about 0.1 to 5 mg/kg body weight about 1 to 5 times, preferably approximately 1 to 3 times a day. For oral administration, the corresponding dose may be administered. When symptoms are extremely serious, the dose may be increased depending on the conditions.

A suitable dose for 24 hours may correspond to the IgY content of 0.05-10 egg yolk from an immunised bird such as an immunised hen or chicken.

A composition according to the invention may comprise 0.1-99 weight %, such as 0.1-99 weight %, 0.1-95 weight %, 0.1-90 weight %, 0.1-85 weight %, 0.1-80 weight %, 0.1-75 weight %, 0.1-70 weight %, 0.1-65 weight %, 0.1-60 weight %, 0.1-55 weight %, 0.1-50 weight %, 0.1-45 weight %, 0.1-40 weight %, 0.1-35 weight %, 0.1-30 weight %, 0.1-25 weight %, 0.1-20 weight %, 0.1-20 weight %, 0.1-15 weight %, 0.1-10 weight %, 0.1-05 weight % of any of the above mentioned Candida and/or Aspergillus species based on the total weight of IgY in the composition, such as of the Candida albicans, Candida glabrata, Candida krusei and Candida parapsilosis

During their experiments, the present inventors have found that the combination of human breast milk and IgY, administered as an emulsion, protects the IgY and conserves its activity. The present inventors believe that this is due to the emulsion that is formed from IgY and the milk. This can also be an effect of the buffering ability of the milk, which enhances the lifetime of the IgY.

The invention also relates to a diagnostic method of fungi diseases, characterised in that blood or tissues from an individual is tested with at least one IgY antibody and a binding reaction is an indication of presence of fungi against which the IgY antibody is directed.

While the invention has been described in relation to certain disclosed embodiments, the skilled person may foresee other embodiments, variations, or combinations which are not specifically mentioned but are nonetheless within the scope of the appended claims.

All references cited herein are hereby incorporated by reference in their entirety.

The expression “comprising” as used herein should be understood to include, but not be limited to, the stated items.

The invention will now be described by way of the following non-limiting examples.

### EXAMPLES

### Example 1

Preparation of Fungi

Candida species isolated from infected patients were used in an in vitro experiment to demonstrate the prophylactic potential of egg immune globulin isolated from domestic hens hyper-immunised with fungi antigen.

The fungi were grown in 500-ml flasks containing 100 ml of 2% glucose, 0.15% yeast nitrogen base, 0.5% ammonium sulphate supplemented with amino acids. The flasks were shaken at 200 r.p.m. in a rotary incubator at 37° C. for 24 hours. The fungi were also grown on candida culture plates used for detection of candia colonisation in patients samples.

Preparation of Anti-Candida IgY Immune Globulin

Suspensions of formalin-killed Candida albicans, Candida glabrata, Candida krusei and Candida parapsilosis was washed in saline and frozen. Each candida specie was used for immunization of a separate group of hens. 10⁷ candida were used per hen per immunization. White leghorn hens were immunized intramuscularly in the breast muscle. For immunization 0.5 mL of candida suspension was mixed with an equal volume of Freunds adjuvant. After the initial immunization, the hens received 2 booster immunizations with 3 week intervals. Yolks of eggs collected from hyper-immunised hens were assayed to determine peak antibody titre using an ELISA (enzyme linked immunosorbent assay) specific for candida-IgY.

When peak titre had been achieved (after 3 immunizations), the egg yolks were harvested by separation from the egg white from the egg yolk. The immune globulin fraction was purified using the water extraction method (Akita E M, Nakai S. Immunoglobulins from egg yolk: isolation and purification. J Food Scie 1992;57:629-634.). Briefly, the yolk was separated from the white and diluted with deionized water at a 1:9 ratio. After storing at 4° C. for at least 6 hr, the supernatant containing the IgY was filtered and frozen at 20° C. The antibodies were also purified by the polyethylene glycol method. Briefly, one part egg yolk was mixed with 2 parts 0.9% NaCl containing 5% PEG6000. The antibody preparation was centrifuged at 2000 g for 30 min at 4° C. After centrifugation solid PEG6000 was added to the supernatant to a final concentration of 12%. The mixture was centrifuged at 2000 g for 30 min at 4° C. and the supernatant was removed. The antibody containing precipitate was dissolved in 0.9% NaCl leaving the proteinaceous polyclonal immune globulin in a purified state. The immune globulin fraction was diluted using 0.9% NaCl to a concentration of 10 mg/ml and frozen. This solution was used to evaluate the prophylactic potential of the anti-Candida IgY indicated below in the example Treatment of leucemic children with anti-candida IgY.

Example 2

Cell Adhesion Assay

Adhesion to mucus epithelium cells is considered to be the primary stage in infection. In this example epithelial cells and pseudomonas aeruginosa (as representative for bacteria) were used to evaluate adhesion.

Fresh cells cultured for 24 hour were washed by centrifugation in PBS, re-suspended in PBS and mixed with medium containing either IgY of eggs from hens immunised with pseudomonas or IgY from non-immunised hens at a ratio of 100:1 or no IgY at all and incubated at 37° C. for 2 hours. After incubation, the culture was filtered through a 45 gm filter to remove any unadhered cells, washed in PBS and re-suspended.

The adherence was evaluated by microscopic examination at 400 magnifications using a stage micrometer grid to facilitate accurate counting. Adherence was expressed as a percentage of cells with visibly adhering pseudomonas aeruginosa bacteria.

The results showed that the adhesion of P. aeruginosa to epithelial cells was reduced by more than 50% in the case of bacteria treated with immune globulin from immunised hens, when compared with both untreated bacteria and bacteria treated with extract of normal egg.

The above in vitro experiments demonstrate that purified immune globulin fractions extracted from the yolk sac of eggs laid by hens previously hyper immunised with P. aeruginosa antigen inhibit epithelial cell adhesion. These experiments enables one to conclude that specific egg immune globulin can be used in the prophylaxis or therapy of infections in epithelial cells.

In Vitro Tests for Activity Against Fungi with Lowered Sensitivity or Resistance to Antimycotics

The Candida glabrata, Candida krusei species used for immunization and testing were resistant to antimycotics. 96-well microtitre plates (F96 Polysorp, Nunc, Roskilde, Denmark) were coated with suspensions of formalin-killed Candida albicans, Candida glabrata, Candida krusei and Candida parapsilosis (the same strains that were used for immunizations diluted 1:1000 in 0.1 M NaHCO₃, pH 9.5 for 2 h at RT or over night at 4° C. Thereafter the wells were washed with 0.02 M NaH₂PO₄, 0.15 M NaCl, pH 7.2 (PBS) containing 0.05% Tween 20 (PBS-T) three times and blocked with 125 μL/well of 3 mg bovine serum albumin/mL in 0.1 M NaHCO₃, pH 9.5 for 1 h on an orbital shaker at RT or over night at 4° C. 100 μL of each antibody preparation diluted in PBS-T were added in duplicates after washing as previously described, and plates were incubated for 1 h at RT. The plates were washed again as above and incubated for 1 h at RT with 100 μL horseradish-peroxidase-(HRP) Rabbit Anti-Chicken/Turkey IgG (IgY) (H+L) (Zymed, San Francisco, Calif., USA) diluted 1:2000 in PBS-T. Bound secondary antibodies were detected with 100 μL 3,3′,5,5′-tethramethylbenzidine (TMB) substrate (Zymed). The reaction was stopped after ten minutes with 50 μL 1.8M H₂SO₄. The absorbance was read at 450 nanometers in a microplate reader (Molecular Devices, Sunnyvale, Calif., USA). An ΔA₄₅₀>0.05 above background was considered significant throughout the experiment.

The ELISA results (FIG. 1-4) show that antibodies directed against all 4 strains show strong crossreactivity and strong reactivity with the antimycotic resistant strains of C. glabrata and C. krusei.

Treatment of Patients with Anti-Fungal IgY

Infections caused by fungi are extremely difficult to eradicate and are dangerous if they proceed to a generalised infection.

In a small clinical study anti-Candida IgY was used to treat four children with leukaemia to prevent candida infections. None of these patients got any sign of candida infection, whereas three out of four control children with leukaemia got signs of candida infection.

The invention is not limited to the embodiments described above which may be modified and/or varied without departing from the scope of the invention.

### Example 3

### Freeze-Drying and Concentration of IgY Extract

Double Freeze-Drying

Twenty (20) freeze-drying flasks were each supplied with 4 ml of IgY extract of the invention. 8.5 mg of lactose was added to 10 of the flasks. 34 mg of lactose was added to the remaining 10 flasks. Flasks were subjected to freeze-drying according to standard protocol.

After the drying, 2 flasks of each of the two lactose concentrations were taken aside and labelled “one freeze-drying”. The product in the remaining flasks was dissolved in distilled water according to the schedule below. For each concentration (“high” or “low”) of lactose:
4 ml water was added to two of the flasks
2 ml water was added to two of the flasks
1 ml water was added to two of the flasks
0.5 ml water was added to two of the flasks

The flasks were labelled with the amount of water added (i.e. 4, 2, 1 and 0.5, respectively). The 16 flasks with re-dissolved IgY were subjected to freeze-drying according to standard protocol. The result was a more compact powder. All solutions were freeze-dried successfully.

Concentration and Freeze-Drying of IgY Extract

11 ml of IgY solution was concentrated with ultrafiltration (from Pall, Type Centrasette,) Cut-off 30 KDa until the volume was 5 ml. The solution was then transferred to a vial and freeze-dried separately. The result was a more compact powder.

Large-Scale Concentration and Freeze-Drying

The sample (approx. 14 l) was thawed. A 10 ml reference sample was taken aside before the process was initiated. The sample was first filtered with a deep-filter (Sartopure PP2 capsule, 0.65 μm), and thereafter with a sterile filter (Sartobran P sterile capsule, 0.65 μm+0.45 μm). Reference samples were taken aside after each filtration step. Volume after filtration was 13.2 l. The solution was concentrated with ultrafiltration (cut-off 30 kD) until the volume was approx. 2.8 l corresponding to a concentration factor of 4.7 times. Reference samples of the ultrafiltration flow-though and the concentrate were taken.

Following concentration, a large scale freeze-drying was performed using approx 2.8 l of concentrated IgY extract. The extract was freeze-dried in bulk in aluminium trays with approx. 5 mm column height. Lactose was not added. Freezing temperature: −47° C.; freezing time: 4 hours; Primary freeze drying: temperature interval: −45° C. to +35° C.; time: 26 hours; pressure 190 ubar. Secondary freeze drying: temperature +30° C.; time: 10 hours; final vacuum under 1 ubar The final product was 98 g of IgY powder.

Conclusion: A satisfactory protocol for concentration and freeze-drying of IgY was developed.

### Example 4

### Preparation of Lozenges Comprising IgY

IgY powder prepared according to example 1 was sieved. After sieving the weight of the IgY powder used as starting material was 48.24 g.

Raw Materials:

| | |
|---|---|
| IgY | of the invention |
| Mannitol | FLUKA Medicago art nr 01-0295 |
| Lactose | BDH Medicago art nr 01-0070 |
| Glycine | MERCK Medicago art nr 01-0181 |
| Magnesium Stearate | |
| Peppermint oil | Apoteket (Pharmacy) |

The amounts indicated below are given per tablet.

Recipe 1:

| | |
|---|---|
| IgY | 926.00 mg |
| Mannitol | 866.58 mg |
| Lactose | 433.42 mg |
| Glycine | 50.00 mg |
| Magnesium stearate | 25.00 mg |
| Peppermint oil | 0.02 ml |
| Tablet weight | 2.30 gram |

Attempts to punch tablets with a 20 mm-punch failed due to that the tablet machine maximum capacity was reached resulting in a tablet weight of 1.15 g. Tablets were punched with this weight and were then repulverised in a sieve. A new attempt was made using this tablet bulk mass resulting in a tablet weight of 1.85 g. The tablet punching process was interrupted for the addition of more excipients.

Recipe 2

| | | |
|---|---|---|
| IgY | 463.00 | mg |
| Mannitol | 1175.24 | mg |
| Lactose | 586.83 | mg |
| Glycine | 50.00 | mg |
| Magnesium stearate | 25.00 | mg |
| Peppermint oil | 0.02 | ml |
| Tablet weight | 2.30 | gram |

The excipients were mixed with the granules of Recipe 1 to final concentrations according to Recipe 2. A total of 65 tablets were punched. Weight variation could be somewhat larger than usual due to the fact that the tablet machine was hand-pulled. The tablet bulk mass was insufficient for weight calibration. Approximately 100 tablets are normally processed to calibrate the machine.

Conclusion: Concentrating the IgY extract has made it possible to go from 25 mm to 20 mm tablet diameter. A recipe for a tablet bulk mass containing IgY, which gives firm tablets, was developed. Further optimisation may be useful for achieving a more convenient mass to work with.

1. A composition comprising IgY antibodies against at least two different fungi chosen from Candida albicans, Candida dubliniensis, Candida glabrata, Candida guilliermondii, Candida kefyr, Candida krusei, Candida lusitaniae, Candida milleri, Candida oleophila, Candida parapsilosis, Candida tropicalis and Candida utilis Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor.

2. The composition according to claim 1, wherein the at least two species are chosen from Candida albicans, Candida glabrata, Candida krusei and Candida parapsilosis.

3. The composition according to claim 1, wherein one of the species is Candida albicans.

4. The composition according to claim 1, wherein the antibodies are polyclonal, monoclonal, humanised polyclonal, humanised monoclonal or antigen binding fragments of these antibodies or a mixture thereof.

5. The composition according to claim 1, further comprising pharmaceutically acceptable carriers, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispensing agents, nutritional agents, e. g. human breast milk or a substitution thereof, functional foods, probiotica and bacteria that are not harmful and against which the IgY antibodies are not directed.

6. The composition according to claim 5, wherein the bacteria are probiotic bacteria such as lactic acid bacteria such as bifidobacterium, lactobacilli, e.g. Lactobacillus reuteri and Lactococci.

7. The composition according to claim 5, wherein the nutritional agent is human breast milk or a substitution therefore.

8. The composition according to claim 1, suitable for local, such as vaginal or oral treatment.

9. Composition comprising IgY antibodies against at least two species from fungi chosen from Candida albicans, Candida dubliniensis, Candida glabrata, Candida guilliermondii, Candida kefyr, Candida krusei, Candida lusitaniae, Candida milleri, Candida oleophila, Candida parapsilosis, Candida tropicalis and Candida utilis Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor for the preparation of a pharmaceutical.

10. Composition comprising IgY antibodies against at least two different fungi chosen from Candida albicans, Candida dubliniensis, Candida glabrata, Candida guilliermondii, Candida kefyr, Candida krusei, Candida lusitaniae, Candida milleri, Candida oleophila, Candida parapsilosis, Candida tropicalis and Candida utilis Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor for the preparation of a pharmaceutical for prophylaxis and/or treatment of a condition caused by fungi, such as organisms belonging to the Candida genus or Aspergillus genus, especially of fungi that are less sensitive to antimycotica.

11. The composition according to claim 9, wherein the condition is chosen from conditions caused by Candida overgrowth chosen from dysfunction in glands and organs, such as adrenal and thyroid gland malfunction, cold hands or feet, diabetes mellitus, hypoglycemia, hypothyroidism, impotence, low body temperature; gastrointestinal dysfunction such as bad breath (halitosis), bloating, coating on tongue (oral thrush), constipation, diarrhea, dry mouth, gas, heartburn, indigestion, irritable bowel syndrome, obesity or/and excessive weight loss; psychological and Allergic dysfunctions such as acne, Blurred vision, bronchitis (recurrent), burning or tingling, chemical sensitivity, chest pain, coughing, earaches, hayfever, headaches, hives, muscle aches, pain, weakness and tension, nasal congestion, head tension, numbness, painful, swollen, stiff joints, shortness of breath, sinusitis, sore throats; emotional and mental dysfunctions chosen from ADD, ADHD, anxiety, depression, disorientation, drowsiness, fatigue, feelings of unreality, frequent mood swings, hyperactivity, inability to concentrate, insomnia, irritability, low energy, mental confusion, MS, nervousness, poor memory: skin dysfunctions such as Acne, anal itch, athlete's foot, dandruff, dermatitis, diaper rash, dry skin, eczema, excessive perspiration, facial rash, fungal infection of the nails, hives, Impetigo, jock itch, lupus, psoriasis, tingling and numbness; genitourinary dysfunction chosen from bladder infection (recurrent), burning on urination, cramps, cystis, endometriosis (irregular or painful menstruation), fluid retention (oedema), frequent urination, impotency, infertility, loss of sexual feelings, menstrual irregularities, painful intercourse, PMS, prostatitis, recurrent yeast vaginitis, vaginal burning, itching or discharge.

12. The composition according to claim 11, wherein the condition caused by the Aspergillus genus is aspergillosis such as allergic bronchopulmonary aspergillosis, pulmonary aspergilloma and invasive aspergillosis and colonization of bacteria from the Aspergillus genus in the respiratory tract.

13. Use of at least two different fungi chosen from Candida albicans, Candida dubliniensis, Candida glabrata, Candida guilliermondii, Candida kefyr, Candida krusei, Candida lusitaniae, Candida milleri, Candida oleophila, Candida parapsilosis, Candida tropicalis and Candida utilis Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor for immunisation of birds.

14. A method for treatment of a condition caused by an organism belonging to the Candida genus and/or the Aspergillus genus wherein a composition comprising IgY antibodies against at least two fungi is administrated to an individual in need thereof.

15. The method according to claim 14, wherein the individual is a mammal.

16. The method according to claim 15, wherein the individual is a human being.

17. A diagnostic method for fungi diseases, characterised in that blood or tissues from an individual is tested with composition comprising IgY antibodies against at least two different fungi chosen from Candida albicans, Candida dubliniensis, Candida glabrata, Candida guilliermondii, Candida kefyr, Candida krusei, Candida lusitaniae, Candida milled, Candida oleophila, Candida parapsilosis, Candida tropicalis and Candida utilis Aspergillus fumigatus Aspergillus flavus, Aspergillus niger. Aspergillus clavatus, Aspergillus glaucus group, Aspergillus nidulans, Aspergillus oryzae, Aspergillus terreus, Aspergillus ustus, and Aspergillus versicolor and a binding reaction is an indication of presence of fungi against which the IgY antibodies are directed.
